(19) United States
(12) Patent Application Publication
(10) Pub. No.: US 2025/0205255 A1
(43) Pub. Date: Jun. 26, 2025

(54) PHOSPHOLIPID COMPOSITIONS FOR TREATING INFECTIONS AND INFLAMMATION

(71) Applicant: Celestial Therapeutics, Inc., Irvine, CA (US)

(72) Inventor: Ajay Gupta, Irvine, CA (US)

(21) Appl. No.: 18/940,361

(22) Filed: Nov. 7, 2024

Related U.S. Application Data
(63) Continuation of application No. 18/095,498, filed on Jan. 10, 2023, now abandoned.
(60) Provisional application No. 63/298,192, filed on Jan. 10, 2022.

(51) Int. Cl. A61K 31/6615; A61K 31/20; A61K 31/201

(52) U.S. Cl. CPC A61K 31/6615; A61K 31/20; A61K 31/201

(57) ABSTRACT

A pharmaceutical composition is provided which contains a phospholipid-containing composition which antagonizes toll-like receptors 1, 2, 3, 6, 7, 8 and 10. The phospholipid-containing composition consists of regioisomers and stereoisomers of palmitoyloleoylphosphatidylglycerol (POPG) consisting of at least 80% of 1-palmitoyl-2-oleoyl-sn-glycero-3-phospho-(1′-rac-glycerol) or a salt thereof, and no more than 20% of its regioisomer 1-oleoyl-2-palmitoyl-sn-glycero-3-phospho-(1′-rac-glycerol) or a salt thereof. The phospholipid-containing composition also has enantiomeric enrichment with at least 51% of the dextro rotatory isomer at the sn-2 position of the terminal glycerol moiety.

### CROSS-REFERENCE TO RELATED APPLICATION

This application claims the benefit of priority from U.S. Provisional Patent Application No. 63/298,192, which was filed on Jan. 10, 2022, which has the same title and inventors, and which is incorporated herein by reference in its entirety.

### FIELD OF THE DISCLOSURE

The present disclosure relates generally to antimicrobial phospholipid compositions, and more particularly to methods for generating and administering the same via inhalation, intranasal, intratracheal, intramuscular and intravenous routes.

### BACKGROUND OF THE DISCLOSURE

POPG is a pulmonary surfactant phospholipid that reduces interfacial tension at the air/water interfaces in the alveoli. This prevents the tension from pulling the alveolar membranes in-wards which would collapse them and lead to respiratory distress. (Pubchem, 2021). Pharmaceutical applications of POPG and other surfactant lipids have been developed and are described, for example, in U.S. Pat. No. 8,367,643 (Voelker et al.).

It has been determined that POPG can markedly attenuate inflammatory responses induced by lipopolysaccharide through direct interactions with the Toll-like receptor 4 (TLR4) interacting proteins CD14 and MD-2. CD14 and TLR4 have been implicated in the host response to Respiratory Syncytial Virus (RSV). Treatment of bronchial epithelial cells with POPG significantly inhibited interleukin-6 and interleukin-8 production, as well as the cytopathic effects induced by RSV [Numata M, Chu HW, Dakhama A, Voelker DR. Pulmonary surfactant phosphatidylglycerol inhibits respiratory syncytial virus-induced inflammation and infection. Proc Natl Acad Sci USA. 2010 Jan. 5; 107 (1): 320-5. doi: 10.1073/pnas.0909361107. Epub 2009 Dec. 22. PMID: 20080799; PMCID: PMC2806703].

### SUMMARY OF THE DISCLOSURE

In one aspect, a pharmaceutical composition is provided which comprises a phospholipid-containing composition which antagonizes toll-like receptors 1, 2, 3, 6, 7, 8 and 10; wherein said phospholipid-containing composition consists of regioisomers and stereoisomers of palmitoyloleoylphosphatidylglycerol (POPG) consisting of at least 80% of 1-palmitoyl-2-oleoyl-sn-glycero-3-phospho-(1′-rac-glycerol) or a salt thereof, and no more than 20% of its regioisomer 1-oleoyl-2-palmitoyl-sn-glycero-3-phospho-(1′-rac-glycerol) or a salt thereof; and wherein said phospholipid-containing composition has enantiomeric enrichment with at least 51% of the dextro rotatory isomer at the sn-2 position of the terminal glycerol moiety.

In another aspect, method for treating or inhibiting an infection or inflammation in a subject is provided. The method comprises administering to the subject an amount of a pharmaceutical composition that is effective to treat or inhibit the infection or inflammation. The pharmaceutical composition comprises a phospholipid-containing composition which antagonizes toll-like receptors 1, 2, 3, 6, 7, 8 and 10; wherein said phospholipid-containing composition consists of regioisomers and stereoisomers of palmitoyloleoylphosphatidylglycerol (POPG) consisting of at least 80% of 1-palmitoyl-2-oleoyl-sn-glycero-3-phospho-(1′-rac-glycerol) or a salt thereof, and no more than 20% of its regioisomer 1-oleoyl-2-palmitoyl-sn-glycero-3-phospho-(1′-rac-glycerol) or a salt thereof; and wherein said phospholipid-containing composition has enantiomeric enrichment with at least 51% of the dextro rotatory isomer at the sn-2 position of the terminal glycerol moiety.

### BRIEF DESCRIPTION OF THE DRAWINGS

FIG. 1 is an illustration of the skeletal formula of 1-Palmitoy-2-loleoyl-sn-glycerol-3-phospho-(1′-rac-glycerol) (POPG).

### DETAILED DESCRIPTION

LPS is a major cause of acute lung injury (ALI) and acute respiratory distress syndrome (ARDS). ALI/ARDS is a life-threatening condition caused by inflammation of the lungs and accumulation of fluid in the alveoli, which results in low blood oxygen levels. Given the severity of symptoms associated with many inflammatory conditions, including those affecting the respiratory system, there is a continued need for agents useful in controlling inflammation and thereby preventing and/or treating conditions or diseases associated with inflammation.

It has now been found that the foregoing needs may be met with the compositions and methodologies disclosed herein. In a preferred embodiment, these compositions are pharmaceutical compositions containing regioisomers and stereoisomers of 1-palmitoy-2-loleoyl-sn-glycerol-3-phospho-(1′-rac-glycerol) (POPG) surfactant. This pharmaceutical composition may be utilized for treatment or prophylaxis in various applications (and preferably pulmonary applications) such as, for example, respiratory syncytial virus (RSV), influenza (including subtype H1N1), human rhinovirus infection, allergic asthma, chronic obstructive pulmonary disease (COPD), sepsis, acute lung injury (ALI), cystic fibrosis (CF), and other maladies. The pharmaceutical composition is preferably administered via an oral inhalation route.

More preferably, the pharmaceutical compositions disclosed herein contain a phospholipid-containing composition which is capable of antagonizing TLR1, 2, 3, 6, 7, 8 and 10, and which preferably consists of regioisomers and stereoisomers of POPG consisting of at least 80% of 1-palmitoyl-2-oleoyl-sn-glycero-3-phospho-(1′-rac-glycerol) (sodium salt) and no more than 20% of its regioisomer 1-oleoyl-2-palmitoyl-sn-glycero-3-phospho-(1′-rac-glycerol) (sodium salt). The pharmaceutical composition preferably has some enantiomeric enrichment with at least 51% of the dextro rotatory isomer at sn-2 position of the terminal glycerol moiety, preferably with an optical rotation of +8.8±1.0°. The pharmaceutical composition preferably contains no more than 0.25% of 1,2-dihexadecanoyl-sn-glycero-3-phospho-(1′-rac-glycerol) (sodium salt) (DPPG-Na); no more than 0.15% of 1-octadecanoyl-2-(9Z-octadecenoyl)-sn-glycero-3-phospho-(1′-rac-glycerol) (sodium salt) (SOPG-Na); no more than 0.5% of 1-palmitoyl-2-hydroxy-sn-glycero-3-phosphocholine (P-Lyso-PC); no more than 0.5% of 1-oleoyl-2-hydroxy-sn-glycero-3-phospho-(1′-rac-glycerol) (sodium salt) (P-Lyso-PG, Na); no more than 0.5% of 1-palmitoyl-2-oleoyl-glycero-3-phosphocholine (POPC); and no more than 0.5% of 1-palmitoyl-2-oleoyl-sn-glycero-3-phosphate (sodium salt) (POPA). Furthermore, the composition preferably contains less than 0.2% free fatty acids and less than 2% of total impurities. In addition, the composition preferably contains 49.0-51.0 mol % palmitic acid and 49.0-51.0 mol % oleic acid with less than 1% sum of other fatty acids.

Aside from the above-noted stereochemistry, the compositions described herein may be utilized or formulated in the manner (or with the additives or formulations) described for the analogous compositions described in U.S. Pat. No. 8,367,643 (Voelker), U.S. Pat. No. 8,796,243 (Voelker), U.S. Pat. No. 9,861,649 (Voelker), and U.S. Pat. No. 10,532,066 (Voelker et al.), all of which are incorporated herein by reference in their entirety.

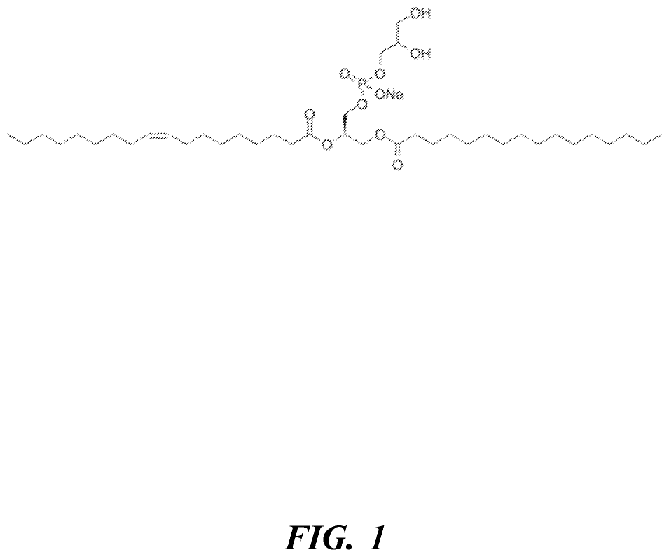

As previously noted, the pharmaceutical compositions disclosed herein preferably contain POPG, the skeletal formula of which is depicted in FIG. 1. POPG is a pulmonary surfactant phospholipid that reduces interfacial tension at the air/water interfaces in the alveoli. This prevents the tension from pulling the alveolar membranes in-wards which would collapse them and lead to respiratory distress.

In a recent study, it was determined that POPG could markedly attenuate inflammatory responses induced by lipopolysaccharide through direct interactions with the Toll-like receptor 4 (TLR4) interacting proteins CD14 and MD-2. CD14 and TLR4 have been implicated in the host response to Respiratory Syncytial Virus (RSV). Treatment of bronchial epithelial cells with POPG significantly inhibited interleukin-6 and interleukin-8 production, as well as the cytopathic effects induced by RSV (Numata, et al., 2009).

The pharmaceutical compositions disclosed herein are preferably administered via an orally inhaled powder. The usage of powder formulations may offer several advantages over other possible administration routes, including greater stability than liquid formulations and a reduced (and possibly zero) need for preservatives. A number of factors such as moisture sensitivity, solubility, particle size, particle shape, and flow characteristics may be considered in tailoring the deposition and absorption properties of these pharmaceutical compositions (see, e.g., Djupesland PG. Nasal drug delivery devices: characteristics and performance in a clinical perspective-a review. Drug Deliv Transl Res. 2013 February; 3 (1): 42-62. doi: 10.1007/s13346-012-0108-9. Epub 2012 Oct. 18. PMID: 23316447; PMCID: PMC3539067).

In a preferred embodiment, a pharmaceutical composition is provided which comprises a phospholipid-containing composition which antagonizes toll-like receptors 1, 2, 3, 6, 7, 8 and 10; wherein said phospholipid-containing composition consists of regioisomers and stereoisomers of palmitoyloleoylphosphatidylglycerol (POPG) consisting of at least 80% of 1-palmitoyl-2-oleoyl-sn-glycero-3-phospho-(l′-rac-glycerol) or a salt thereof, and no more than 20% of its regioisomer 1-oleoyl-2-palmitoyl-sn-glycero-3-phospho-(1′-rac-glycerol) or a salt thereof; and wherein said phospholipid-containing composition has enantiomeric enrichment with at least 51% of the dextro rotatory isomer at the sn-2 position of the terminal glycerol moiety. The phospholipid-containing composition preferably has an optical rotation within the range of +8.8±1.0°.

The phospholipid-containing composition also preferably contains no more than 0.25% of 1,2-dihexadecanoyl-sn-glycero-3-phospho-(1′-rac-glycerol) or a salt thereof, preferably no more than 0.15% of 1-octadecanoyl-2-(9Z-octadecenoyl)-sn-glycero-3-phospho-(I′racglycerol) or a salt thereof; preferably no more than 0.5% of 1-palmitoyl-2-hydroxy-sn-glycero-3-phosphocholine (P-Lyso-PC) or a salt thereof; preferably no more than 0.5% of 1-oleoyl-2-hydroxy-sn-glycero-3-phospho-(l′-rac-glycerol) or a salt thereof, preferably no more than 0.5% of 1-palmitoyl-2-oleoyl-glycero-3-phosphocholine (POPC) or a salt thereof, preferably no more than 0.2% free fatty acids; and preferably less than 2% of total impurities. The phospholipid-containing composition also preferably contains 49.0-51.0 mol % palmitic acid; preferably contains 49.0-51.0 mol % oleic acid; preferably contains less than 1% of other fatty acids; and preferably contains less than 1% of other fatty acids.

The methodologies disclosed herein may be utilized to treat various diseases or indications. Thus, for example, these compositions may be utilized to treat subjects who have, or who are at risk of developing, rhinovirus infections, respiratory Syncytial virus infections, influenza infections, and inflammation.

The above description of the present invention is illustrative and is not intended to be limiting. It will thus be appreciated that various additions, substitutions and modifications may be made to the above described embodiments without departing from the scope of the present invention. Accordingly, the scope of the present invention should be construed in reference to the appended claims. For convenience, some features of the claimed invention may be set forth separately in specific dependent or independent claims. However, it is to be understood that these features may be combined in various combinations and sub combinations without departing from the scope of the present disclosure. By way of example and not of limitation, the limitations of two or more dependent claims may be combined with each other without departing from the scope of the present disclosure.

1-70. (canceled)

71. A pharmaceutical composition, consisting of:
at least 80% of 1-palmitoyl-2-oleoyl-sn-glycerol-phospho-(1′-rac-glycerol) or a salt thereof;
no more than 20% of 1-olcoyl-2-palmitoyl-sn-glycero-3-phospho-(1′-rac-glycerol) or a salt thereof;
no more than 1% of:
1,2-dihexadecanoyl-sn-glycero-3-phospho-(1′-rac-glycerol) or a salt thereof;
1-octadecanoyl-2-(9Z-octadecenoyl)-sn-glycero-3-phospho-(1′-rac-glycerol) or a salt thereof;
1-palmitoyl-2-hydroxy-sn-glycero-3-phosphocholine or a salt thereof;
1-oleoyl-2-hydroxy-sn-glycero-3-phospho-(1′-rac-glycerol) or a salt thereof; and
1-palmitoyl-2-oleoyl-glycero-3-phosphocholine or a salt thereof;
palmitic acid; and
oleic acid.

72. The pharmaceutical composition of claim 71, which is formulated to antagonize toll-like receptors 1, 2, 3, 6, 7, 8 and 10.

73. The pharmaceutical composition of claim 71, wherein at least 51% of the dextro-rotatory isomer of each component is at the sn-2 position of the terminal glycerol moiety.

74. The pharmaceutical composition of claim 71, which has an optical rotation of +8.8±1.0°.

75. The pharmaceutical composition of claim 71, wherein the 1,2-dihexadecanoyl-sn-glycero-3-phospho-(1′-rac-glycerol) or a salt thereof is in an amount of no more than 0.25%.

76. The pharmaceutical composition of claim 71, wherein the 1-octadecanoyl-2-(9Z-octadecenoyl)-sn-glycero-3-phospho-(1′-rac-glycerol) or a salt thereof is in an amount of no more than 0.15%.

77. The pharmaceutical composition of claim 71, wherein the 1-palmitoyl-2-hydroxy-sn-glycero-3-phosphocholine (P-Lyso-PC) or a salt thereof is in an amount of no more than 0.5%.

78. The pharmaceutical composition of claim 71, wherein the 1-oleoyl-2-hydroxy-sn-glycero-3-phospho-(1′-rac-glycerol) or a salt thereof is in an amount of no more than 0.5%.

79. The pharmaceutical composition of claim 71, wherein the 1-palmitoyl-2-oleoyl-glycero-3-phosphocholine or salt thereof is in an amount of no more than 0.5%.

80. The pharmaceutical composition of claim 71, which is free of any free fatty acids.

81. The pharmaceutical composition of claim 71, which is free of impurities.

82. The pharmaceutical composition of claim 71, wherein the palmitic acid is in an amount of 49.0-51.0 mol %.

83. The pharmaceutical composition of claim 71, wherein the oleic acid is in an amount of 49.0-51.0 mol %.

84. The pharmaceutical composition of claim 71, which is formulated as a powder for oral inhalation.
